(19) United States
(12) Plant Patent Application Publication
(10) Pub. No.: US 2023/0210033 P1
(43) Pub. Date: Jun. 29, 2023

(54) Rose Plant Named ‘Poulty029’

(71) Applicant: Mogens Nyegaard Olesen, Fredensborg (DK)

(72) Inventor: Mogens Nyegaard Olesen, Fredensborg (DK)

(21) Appl. No.: 17/803,626

(22) Filed: Sep. 13, 2022

(30) Foreign Application Priority Data
Sep. 24, 2021 (QZ) 2021/2347

(51) Int. Cl. A01H 6/74

(52) U.S. Cl. PLT/139

(57) ABSTRACT

A new garden rose plant of the Miniature class which has abundant, red flowers and attractive foliage. This new and distinct variety has shown to be uniform and stable in the resulting generations from asexual propagation.

Botanical Designation: Rosa hybrida .

Variety Denomination: ‘Poulty029’.

This application claims priority to Plant Breeder’s Rights Application Number 2021/2347, which was filed at the Community Plant Variety Rights Office in the European Union on Sep. 24, 2021, the contents of which are hereby incorporated by reference for all purposes.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2013, and the resulting seeds were planted in a controlled environment in Fredensborg, Denmark. The new variety, named ‘Poulty029’, originated as a single seedling from the stated cross.

The new variety may be distinguished from its male pollen parent and female seed parent by the following characteristics. While both the male pollen parent and the claimed plant have red flowers, the new variety is more compact in height growing to about 37 cm. The pollen parent grows to 45 cm in height. The female seed parent plant has yellow orange flowers while the new variety has red flowers.

The objective of the hybridization of this rose variety was to create a new and distinct variety with unique qualities, such as:
1. Uniform and abundant red flowers with long lasting quality;
2. Vigorous, but compact growth when propagated on its own roots;
3. Exceptional disease resistance.

This combination of qualities is not present in previously available commercial cultivars of this type, known to the inventor, and distinguish ‘Poulty029’ from all other varieties of which we are aware.

As part of the rose development program, Mogens N. Olesen germinated the seeds from the aforementioned hybridization during winter of 2013 and conducted evaluations on the resulting seedlings in a controlled environment in Fredensborg, Denmark. ‘Poulty029’ was selected in the spring of 2014 by the inventor as a single plant from the progeny of the aforementioned hybridization.

Asexual reproduction of ‘Poulty029’ by rooted cuttings was first done by Mogens N. Olesen in the nursery in Fredensborg, Denmark in July, 2014. This initial and other subsequent asexual propagations conducted in controlled environments have demonstrated that the characteristics of ‘Poulty029’ are true to type and are transmitted from one generation to the next.

### DESCRIPTION OF THE DRAWING

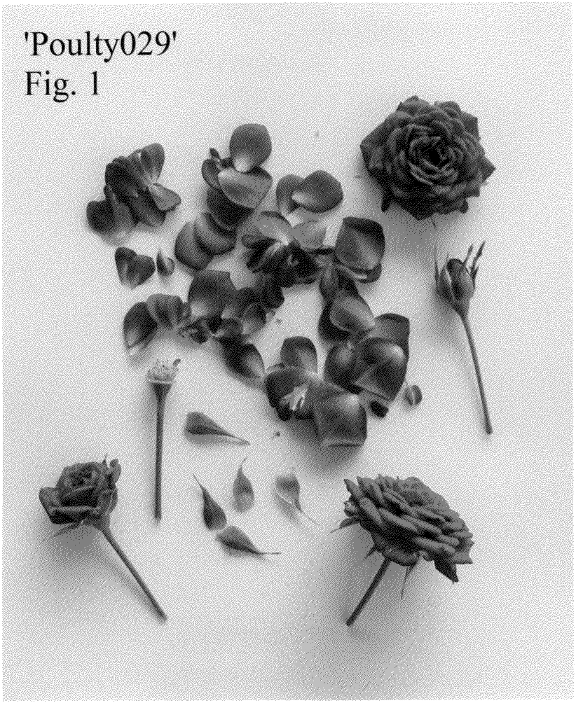

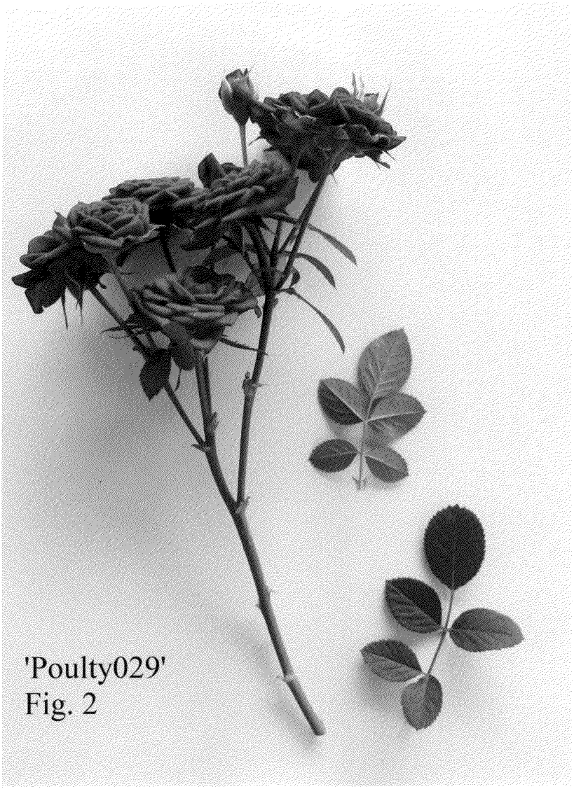

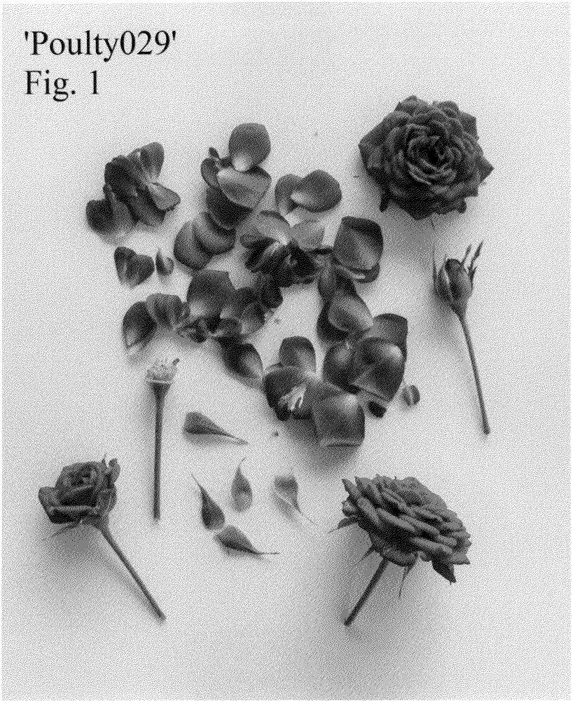

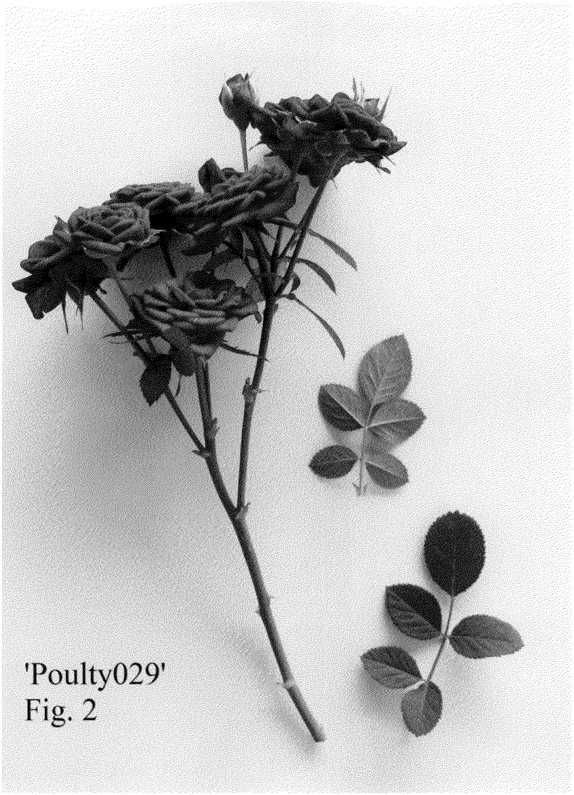

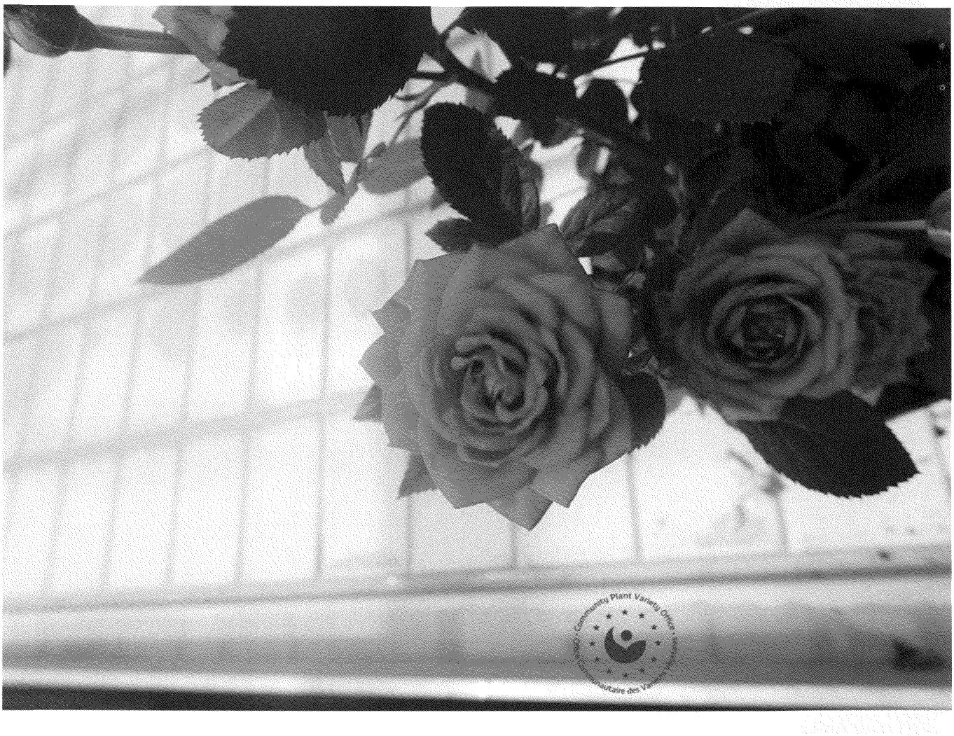

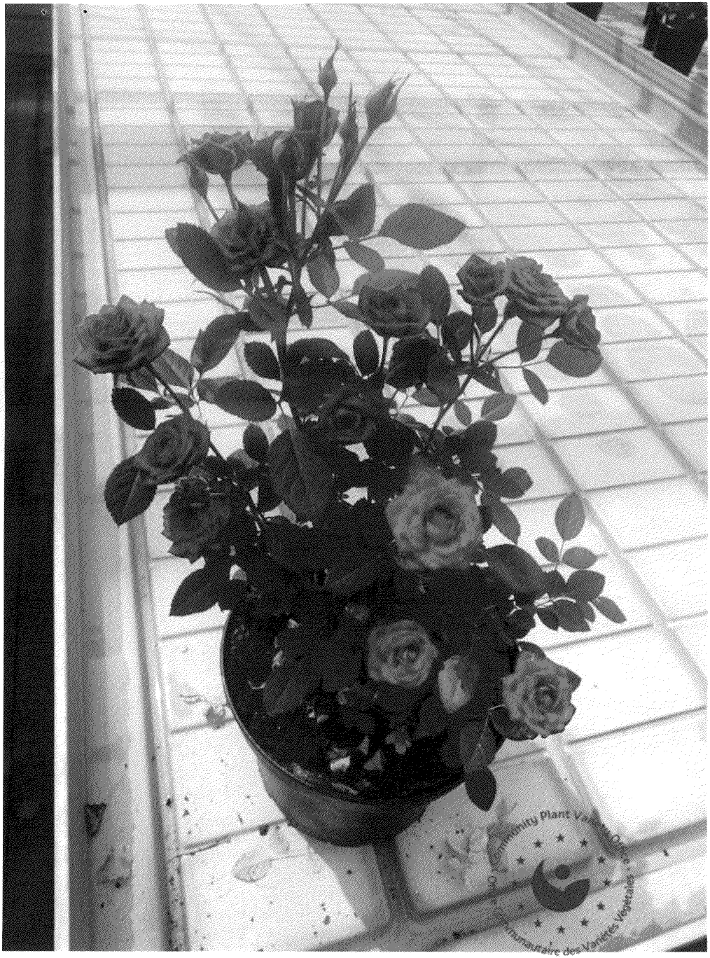

The accompanying color illustrations show as true as is reasonably possible to obtain in color photographs of this type, the typical characteristics of the buds, flowers, leaves, and stems, of ‘Poulty029’. Specifically illustrated in FIG. 1 of the drawings are open flowers at various stages of development viewed from above and the side, flower petals detached revealing reproductive flower parts, sepals detached showing receptacle, and a flower bud as the sepals begin to divide. Specifically shown in FIG. 2 is a cluster of flowers on a branch, bare stem exhibiting prickles, and leaves. Plants shown are 6 months of age.

### DETAILED DESCRIPTION OF THE VARIETY

The following is a description of ‘Poulty029’,as observed in its growth in a glass house located in Odense Denmark. Observed plants are 6 months of age, and were grown on their own roots in 24 cm containers. Color references are made using the Royal Horticultural Society (London, England) Colour Chart, 2001, except where common terms of color are used.

For a comparison, several physical characteristics of the rose variety ‘Poulty011’, U.S. Plant Pat. Number 25,296 are now compared to ‘Poulty029’. While the flower general tonality of the comparison is Red Group 45B the new variety is Red Group 53B. The comparison variety has a flower petal count of 30, while the new variety has 70 petals.

### FLOWER AND FLOWER BUD

Blooming habit: Continuous.
Flower bud:
   Size.—Upon opening, 20 mm in length from base of receptacle to end of bud. Bud diameter is 11 mm.
   Bud form.—Globose.
   Bud color.—As sepals divide petals are Red-Purple Group 59A.
   Sepal inner surface.—Color: Green Group 138B. Surface: Lightly pubescent.
   Sepal outer surface.—Color: Yellow-Green Group 144A. Texture: Smooth.
   Sepal Shape.—Apex: Cirrhose. Base: Flat at union with receptacle.
   Sepal Margin.—Margins have weak to no foliaceous appendages on three of the five sepals.
   Sepal size.—About 20 mm long, 6 mm wide.
   Receptacle.—Texture: Smooth. Size: 5 mm in height, 7 mm wide. Color: Yellow-Green Group 144A. Shape: Funnel.
   Pedicel.—Surface: Smooth with some stipitate glands. Length: 40 mm average. Diameter: 2 mm on average. Color: Yellow-Green Group 144A. Strength: Strong.
   Peduncle.—Length: 30 to 65 mm. Diameter: About 3 mm. Color: Yellow-Green Group 144A. Texture: Smooth.
Flower bud development: Flower buds are borne in clusters of 5 to 7 flower buds per stem, on average. Development as a panicle.
Flower bloom:
   Fragrance.—None.
   Duration.—The blooms have a duration on the plant of approximately 25 days. Petals fall cleanly away from plant after flowers have fully matured.
   Size.—Flower diameter is 45 mm when open. Flower depth is 15 mm.
   Flower shape.—Rosette, very double flower, with many slightly overlapping petals of different sizes.
   Shape of flower, side view.—The upper portion is convex. The lower portion is flat concave. .
Petalage: Under normal conditions, flowers have about 70 petals.
General Tonality of Flower: Open flowers are Red Group 53B. Color remains as flowers mature.
Petal Color:
   Upon opening, outer and inner petals.—Upper surface: Red Group 46A, splashed with Red Group 56D. Basal zone marked with Yellow Group 2C. Lower surface: Red Group 53A. Yellow Group 1C at basal zone.
   After opening, outer and inner petals.—Upper surface: Red Group 53A. Basal zone White Group N155A. Lower surface: Red-Purple Group 60A. Basal zone White Group N155B.
Petals:
   Petal Reflex.—Strong to moderate reflex.
   Margin.—Entire and uniform. Moderate undulations.
   Shape: Broad and elliptic.—Apex shape: Rounded. Base shape: Acute.
   Size.—24 mm (1) x 25 mm (w).
   Texture.—Smooth.
   Thickness.—Average.
Petaloids:
   Size.—8 mm (1) by 5 mm (w).
   Quantity.—About 10.
   Shape.—Elliptical with an acute base and rounded apices.
   Color.—The upper side is Red Group 46A, with Yellow Group 2C at the basal zone. The lower surface is Red Group 53A with a petal spot of Yellow Group 1C at basal zone.
Reproductive Flower Parts:
   Pollen.—None observed.
   Anthers.—Size: 1.5 mm in length. Color: Yellow Group 9B. Quantity: 30 on average.
   Filaments.—Color: Yellow Group 12B. Length: 3 mm.
   Pistils.—Length: 6 mm. Quantity: 20 on average.
   Stigmas.—Color: Green-White Group 157A.
   Styles.—Color: Green-White Group 157A.
   Location of stigmas.—Superior in location relative to the length of the filaments and the height of the anthers.
   Hips.—None Observed.

### PLANT

Plant growth: Upright, bushy, very compact. Plants are 37 cm in height, and 30 cm wide. .
Stems:
   Color of juvenile growth.—Yellow-Green Group 144B.
   Color of mature growth.—Yellow-Green Group 144A.
   Length.—Canes are about 18 cm from the base of the plant to the flowering portion.
   Diameter.—About 4 mm.
   Internodes.—On mature canes about 30 mm between nodes.
   Surface Texture.—Young wood: Smooth. Older wood: Smooth.
Long Prickles:
   Incidence.—About 3 prickles per 10 cm of stem.
   Size.—Average length of prickles on mature stems is 4 to 5 mm.
   Shape.—Upper portion is linear. Lower portion is concave.
   Color.—Juvenile prickles: Greyed-Yellow Group 161D. Mature prickles: Greyed-Yellow Group 161D. .
Plant foliage:
   Compound leaf.—65 to 70 mm (1) x 42 (w).
   Quantity.—3 leaves per 10 cm of stem on average.
   Leaf bearing angle to the stem.—45 degrees.
   Color of juvenile foliage.—Upper side: Yellow-Green Group 144A. Lower side: Yellow-Green Group 144B.
   Color of mature foliage.—Upper side: Yellow-Green Group 147A. Lower side: Yellow-Green Group 147B.
Plant leaves and leaflets:
   Stipules.—Size: 11 mm long, 2 mm wide. Quantity: 2 per compound leaf. Shape: Linear, slightly broad based with outward extending apices. Margins: Finely serrated. Color: Yellow-Green Group 144A.
   Petiole.—Length: 20 mm. Diameter: 2 mm. Upper surface color: Yellow-Green Group 144A. Lower surface color: Yellow-Green Group 144A.
   Rachis.—Length: About 35 mm. Upper surface color: Yellow-Green Group 144A. Lower surface color: Yellow-Green Group 144A.
   Leaflet.—Quantity: Normally 5 leaflets. Margins: Serrated. Size: Terminal leaflets are about 27 mm long, 20 mm wide. Shape: Generally elliptical. Base: Rounded. Apex: Acute. Texture: Smooth. Thickness: Average. Arrangement: Odd pinnate. Venation: Reticulate. Glossiness: Not glossy. .
Disease resistance: Above average resistance to powdery mildew Sphaerotheca pannosa var. rosae, downy mildew Peronospora sparsa, rust Phragmidium spp., black spot Diplocarpon rosae, and Botrytis cinerea under normal growing conditions. .
Cold Hardiness: The variety is tolerant to USDA Cold Hardiness Zone 6. .
Heat Tolerance: The variety has been found to be suitable for climate conditions found in the American Horticulture Society heat zone 7.

I claim:

1. A new and distinct variety of rose plant named ‘Poulty029’ substantially as described and illustrated herein.
